(19) United States
(12) Patent Application Publication
(10) Pub. No.: US 2006/0169529 A1
(43) Pub. Date: Aug. 3, 2006

(54) Diagnosis assist system, data processing terminal and data processing program

(76) Inventor: Yasuaki Tamakoshi, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/563,427

(22) PCT Filed: Jul. 8, 2004
(86) PCT No.: PCT/JP04/10084

§ 371 Date: Jan. 5, 2006

(30) Foreign Application Priority Data
Jul. 10, 2003 (JP) 21003-194970

(51) Int. Cl. A61B 7/02

(52) U.S. Cl. 181/131

(57) ABSTRACT

In the data processing terminal 30a, when the body sound data is generated by the data processing section 38, the body sound ID is given to the concerned body sound data by the control section 31. Then, when the patient ID, operator ID of the operator, sound collector ID of the used sound collector, are inputted through the input section 32 as the additional information of the body sound, the information of the sound collection date and time and the information of the sound collection position of the sound collector M are acquired by the control section 31 when the body sound is sound-collected. When the body sound data is stored in DB 20, each information of the body sound ID, patient ID, operator ID, sound collector ID, sound collection date and time, and sound collection position, is corresponded, and written in the body sound registration table 26.

### FIELD OF THE INVENTION

The present invention relates to a medical examination support system by which data processing of an body sound collected from a medical examination part of a patient is conducted, a data processing terminal and a data processing program.

### BACKGROUND OF THE INVENTION

Conventionally, when the medical examination of heart-lung system of the patient is conducted, there is developed a system by which a respiratory sound or a cardiac sound of the patient is collected by using a stethoscope provided with a sound-collection section such as a microphone, and when it is stored in a server as a digital acoustic signal, the digital acoustic signal can be reproduced by a doctor or the patient at any time (for example, refer to Patent Document 1, Patent Document 2).

(Patent Document 1)
Tokkai No. 2003-93381

(Patent Document 2)
Tokkai No. 2001-327488

In the conventional medical examination, only a present situation whether the respiratory sound is normal or abnormal is examined from a respiratory sound of the patient, or when it is abnormal, by which symptoms it is abnormal, is examined, however, it is not considered that the medical examination is conducted by comparing the respiratory sound of the past with the present one. Therefore, even when the past respiratory sound of the same patient is stored, a system which is suitable for judging a progress situation or recovery situation of disease by comparing the body sound of the past with the present one, is not proposed.

The object of the present invention is to provide a medical examination support system in which the body sound data of the patient are stored as a data-base, and by which a comparison result in which the stored plurality of different body sound data are compared, can be displayed.

### DISCLOSURE OF THE INVENTION

The invention written in item 1 is characterized in that: it is a medical examination support system structured by including: a sound collection means for collecting an body sound of a patient; data processing terminal for data processing the body sound data of the patient; and data base connected to the data processing terminal, and the data processing terminal is provided with: a data preservation means which corresponds the body sound data of the body sound collected by the sound collection means with the identification information of the patient, and stores them in the data base; an input means for inputting the identification information of the patient who is a stethoscopy object; an acquiring means for acquiring the body sound data corresponding to the inputted identification information of the patient from the data base; and a comparison result display means for displaying the comparison result in which different plurality of body sounds data are compared with each other.

The invention written in item 11 is characterized in that: in the data processing terminal, it is provided with: a data preservation means for preserving in the data base by corresponding the body sound data of the body sound collected by a sound collection means for collecting the body sound of the patient with the identification information of the patient; an input means for inputting the identification information of the patient who is a stethoscopy object; an acquiring means for acquiring the body sound data corresponding to the inputted identification information of the patient from the data base; and a comparison result display means for displaying the comparison result in which a plurality of different body sound data are compared with each other.

The invention written in item 21 is characterized in that: it is a data processing program for making a computer realize: the data preservation function for preserving in the data base by corresponding the body sound data of the body sound collected by a sound collection means for collecting the body sound of the patient with the identification information of the patient; the acquiring function for acquiring the body sound data corresponding to the inputted identification information of the patient from the data base; and the comparison result display function for displaying the comparison result in which a plurality of different body sound data are compared with each other.

According to the invention written in items 1, 11, 21, because the body sound data of the patient is corresponded with the identification information of the patient and stored in the data base, and the body sound data corresponding to the patient is acquired from the data base by using the identification information of the patient, when stethoscoped by reading the body sound data stored in the data base, the patient to be stethoscoped can be specified and even when there are a plurality of patients to be stethoscoped, it can be judged which patient has the concerned body sound.

Accordingly, a mistake of the body sound data of the patient can be prevented, and because the comparison result of the adequate plurality of body sound data can be displayed, the high reliable examination support can be conducted. Hereupon, the comparison result may also be shown by a sound such as a difference sound or an announce, or may also be displayed by a visible display means such as a monitor by a sentence or a sign or a graph. Further, the comparison result may also be reproduced by inputting a sound for urging an attention at a timing to be noticed during the stethoscopic sound reproduction or a mark may also be displayed by a display means which can be visibly confirmed during the stethoscopic sound reproduction.

The invention written in item 2 is characterized in that: in the medical examination support system written in item 1, in the data processing terminal, it is provided with an body sound display means for visually and comparably displaying a plurality of different body sound data.

The invention written in item 12 is characterized in that: in the data processing terminal written in item 11, it is provided with an body sound display means for visually and comparably displaying a plurality of different body sound data.

The invention written in item 22 is characterized in that: in the data processing program written in item 21, it includes a function for visually and comparably displaying a plurality of different body sound data on the body sound display means.

According to the inventions written in items 2, 12, 22, because a plurality of different body sound data are comparably displayed, the stethoscope person can visibly compare the body sound data for example, in the past and the present, and the body sound data in the left chest and the right chest, and can judge the progress condition or recovery condition of a disease. Hereupon, the body sound display means may also be integrated with the comparison result display means, or may also be a separated one. Further, when the body sound display means is integrated with the comparison result display means, it may also be a form in which the body sound display means and the comparison result display means are switched, or may also be a form in which the body sound display means and the comparison result display means are simultaneously satisfied.

The invention written in item 3 is characterized in that: in the medical examination support system written in item 1 or 2, in the data processing terminal, it is provided with a comparison means by which a plurality of different body sound data are compared and the comparison result is outputted to the comparison result display means.

The invention written in item 13 is characterized in that: in the data processing terminal written in item 11 or 12, it is provided with the comparison means by which a plurality of different body sound data are compared and the comparison result is outputted to the comparison result display means.

The invention written in item 23 is characterized in that: in the data processing program written in item 21 or 22, it includes a comparison function by which a plurality of different body sound data are compared and the comparison result is outputted to the comparison result display function.

According to the inventions written in items 3, 13, 23, because the data processing terminal compares a plurality of different body sound data and outputs the comparison result, the calculation load on the data base side is decreased.

The invention written in item 4 is characterized in that: in the medical examination support system written in item 2 or 3, the plurality of different body sound data are body sound data of a specific patient, and the body sound data acquired from the data base by using the identification information of the patient, is included.

The invention written in item 14 is characterized in that: in the data processing terminal written in item 12 or 13, the plurality of different body sound data are body sound data of a specific patient, and the body sound data acquired from the data base by using the identification information of the patient, is included.

The invention written in item 24 is characterized in that: in the data processing terminal written in item 22 or 23, the plurality of different body sound data are body sound data of a specific patient, and the body sound data acquired from the data base by using the identification information of the patient, is included.

According to the invention written in items 4, 14, 24, the medial examination support can be conducted by acquring a plurality of body sound data of the same patient, and it contributes to an efficiency increase of the medical examination. Hereupon, the plurality of different body sound data may also be the body sound data acquired from the data base and may also be an body sound data before preserving it in the data base, which is just generated after the sound collection. In this case, the present body sound which is just collected can compare to the past body sound, and it contributes to an increase of the medical examination efficiency by a rapid medical examination support. Further, when the data processing terminal can store a plurality of body sound data, a plurality of different body sound data may also be the plurality of body sound data of the same patient acquired from the data base. In this case, it may also be the comparison of the body sound data stored recently to the past body sound data, or the comparison of mutual past body sound data.

The invention written in item 5 is characterized in that: in the medical examination support system written in any one item of items 1-4, it is provided with a sound reproduction means for reproducing the body sound based on the body sound data acquired by the acquiring means.

The invention written in item 15 is characterized in that: in the data processing terminal written in any one item of items 11-14, it makes the reproduction means reproduce the body sound based on the body sound data acquired by the acquiring means.

The invention written in item 25 is characterized in that: in the data processing program written in any one item of items 21-24, it includes a sound reproduction function for making the sound reproduction means reproduce the body sound based on the body sound data acquired by the acquiring means.

According to the inventions written in items 5, 15, 25, because the body sound data is acquired from the data base and the body sound is reproduced, the stethoscope person can stethoscope the past body sounds stored in the data base. Accordingly, when the past or present body sounds which are desired to compare are successively reproduced, it can be aurally compared.

The invention written in item 6 is characterized in that: in the medical examination support system written in any one item of items 1-5, in the data processing terminal, the input means inputs the identification information of the sound collection means when the body sound is collected by the sound collection means, and the data preservation means stores the identification information of the sound collection means inputted as the additional information of the body sound data in the data base.

The invention written in item 16 is characterized in that: in the data processing terminal written in any one item of items 11-15, the input means inputs the identification information of the sound collection means when the body sound is collected by the sound collection means, and the data preservation means stores the identification information of the sound collection means inputted as the additional information of the body sound data in the data base.

The invention written in item 26 is characterized in that: in the data processing terminal written in any one item of items 21-25, in the case where the body sound is collected by the sound collection means, when the identification information of the sound collection means is inputted through the input means, the data preservation function stores the identification information of the sound collection means inputted as the additional information of the body sound data in the data base.

According to the inventions written in items 6, 16, 26, because the identification information of the sound collection means is stored in the data base as the additional information of the body sound data, it can be easily judged by which sound collection means the body sound data is collected. For example, correspondence that the body sound data relating to the sound collection means in which a trouble is discovered later, is not used for the stethoscopy, can be taken, and the adequate support of the diagnosis can be conducted.

The invention written in item 7 is characterized in that: in the medical examination support system written in any one item of items 1-6, in the data processing terminal, when the body sound is collected by the sound collection means, the input means inputs the identification information of the operator who operates the sound collection operation of the body sound, and the data preservation means stores the inputted identification information of the operator which is inputted as the additional information of the body sound data, in the data base.

The invention written in item 17 is characterized in that: in the data processing terminal written in any one item of items 11-16, when the body sound is collected by the sound collection means, the input means inputs the identification information of the operator who operates the sound collection operation of the body sound, and the data preservation means stores the inputted identification information of the operator which is inputted as the additional information of the body sound data, in the data base.

The invention written in item 27 is characterized in that: in the data processing program written in any one item of items 21-26, in the case where the body sound is collected by the sound collection means, when the identification information of the operator who operates the sound collection operation of the body sound is inputted through the input means, the data preservation means stores the inputted identification information of the operator which is inputted as the additional information of the body sound data, in the data base.

According to the inventions written in items 7, 17, 27, because the identification information of the operator is stored in the data base as the additional information of the body sound data, the body sound data can be acquired from the data base based on the identification information of the operator, and for example, when the body sound data are compared, the body sound data collected by the same operator are acquired and the medical examination for which the operation characteristic different by the operator is considered, can be conducted.

The invention written in item 8 is characterized in that: in the medical examination support system written in any one item of items 1-7, it is provided with a position detection means for detecting the position of the sound collection which is sound-collected by the sound collection means, and the data preservation means of the data processing terminal stores the information of the sound collection position detected as the additional information of the body sound data, in the data base.

The invention written in item 18 is characterized in that: in the data processing terminal written in any one item of items 11-17, the data preservation means stores the information of the sound-collection position detected as the additional information of the body sound data, in the data bases when the sound-collection position is detected by the position detection means by which the sound-collection position collected by the sound collection means is detected.

The invention written in item 28 is characterized in that: in the data processing program written in any one item of items 21-27, the data preservation function stores the information of the sound-collection position detected as the additional information of the body sound data, in the data base, when the sound-collection position is detected by the position detection means by which the sound-collection position collected by the sound collection means is detected.

According to the inventions written in items 8, 18, 28, because the information of the sound-collection position of the body sound is stored as the additional information of the body sound data in the data base, the stethoscope person can easily grasp the sound, of which medical examination part the body sound is.

The invention written in item 9 is characterized in that: in the medical examination support system written in any one item of items 1-8, in the data processing terminal, it is provided with a timer means for timing the date and time at which the sound collection is conducted by the sound collection means, and the data preservation means stores the information of the sound collection date and time timed by the timer means as the additional information of the body sound data in the data base.

The invention written in item 19 is characterized in that: in the data processing terminal written in any one items 11-18, it is provided with a timer means for timing the date and time at which the sound collection is conducted by the sound collection means, and the data preservation means stores the information of the sound collection date and time timed by the timer means as the additional information of the body sound data in the data base.

The invention written in item 29 is characterized in that: in the data processing program written in any one item of items 21-28, it includes a timer function for timing the date and time at which the sound collection is conducted by the sound collection means, and the data preservation function stores the information of the sound collection date and time timed as the additional information of the body sound data in the data base.

According to the invention written in items 9, 19, 29, because the information of the sound collection date and time is stored as the additional information of the body sound data in the data base, the stethoscope person can easily grasp when the body sound is sound-collected.

The invention written in item 10 is characterized in that: in the medical examination support system written in any one item of items 6-9, in the data processing terminal, when the body sound data is acquired from the data base, the input means designation-inputs the additional information of any one of the identification information of the sound-collection means, identification information of the operator, information of the sound-collection position, information of the sound-collection date and time, and the acquiring means acquires the body sound data corresponding to the additional information designation-inputted by the input means from the data base.

The invention written in item 20 is characterized in that: in the data processing terminal written in any one item of items 16-19, when the body sound data is acquired from the data base, the input means designation-inputs the additional information of any one of the identification information of the sound-collection means, identification information of the operator, information of the sound-collection position, information of the sound-collection date and time, and the acquiring means acquires the body sound data corresponding to the additional information designation-inputted by the input means from the data base.

The invention written in item 30 is characterized in that: in the data processing program written in any one item of items 26-29, in the case where the body sound data is acquired from the data base, when the additional information of any one of the identification information of the sound-collection means, identification information of the operator, information of the sound-collection position, information of the sound-collection date and time, is designation-inputted through the input means, the acquiring function acquires the body sound data corresponding to the additional information designation-inputted by the input means from the data base.

According to the inventions written in items 10, 20, 30, because the body sound data corresponding to the additional information is acquired based on the additional information, the stethoscope person can acquires the body sound data desired to stethoscope when any one of the identification information of the sound-collection means, identification information of the operator, information of the sound-collection position, information of the sound-collection date and time, is designated. Accordingly, the body sound data can easily be searched and an increase of the medical examination efficiency can be intended.

### BRIEF DESCRIPTION OF THE DRAWINGS

FIG. 1 is a view showing a system structure of a medical examination support system 100 of an embodiment to which the present invention is applied.

FIG. 2(a) is a view showing an example of a data structure of a DB 20, and FIG. 2(b) is a view showing an example of a data structure of an body sound registration table 26.

FIG. 3 is a sketch drawing of a data processing terminal 30a.

FIG. 4 is a view showing an internal structure of the data processing terminal 30a.

FIG. 5 is a view for explaining a sound collection position by a sound collector M.

FIG. 6 is a flowchart for explaining an body sound preservation processing executed by data processing terminals 30a-30c.

FIG. 7(a) and FIG. 7(b) are image plane transition views displayed in a display section 33 at the time of the body sound preservation processing.

FIG. 8 is a flowchart for explaining an body sound reproduction processing executed by data processing terminals 30a-30c.

FIG. 9(a) and FIG. 9(b) are image plane transition views displayed in the display section 33 in the body sound reproduction processing.

FIG. 10(a) and FIG. 10(b) are image plane transition views displayed in a display section 33 in the body sound reproduction processing.

### BEST MODE FOR CARRYING OUT THE INVENTION

Referring to drawings, embodiments of the present invention will be described below. Hereupon, in the description in the column of the embodiment of the invention, although there is conclusive expressions, they specify the best mode of the invention, and do not limit the scope of the present invention or do not specify the meaning of terminology written in the scope of Claims.

In the present embodiment, an example in which, when the body sound data is stored in the data base, as the additional information of the body sound data, the identification information of a patient whose body sound is collected, identification information of the operator who conducts the sound-collection, identification information of the sound-collector, sound-collection position information and the information of sound collection date and time are corresponded and registered, and when the body sound is reproduced, a plurality of body sound data are read from the data base based on these accompanied information, and the body sound data are comparably displayed, will be described.

Hereupon, as the body sound, for example, a respiratory sound in the chest of the patient, lungs sound (including the bronchial sound) or the cardiac sound of the heart, an intestine noise in the abdominal region (called also GURU-sound), Colotocoph sound of the blood vessel in the wrist, are listed.

Initially, the structure will be described.

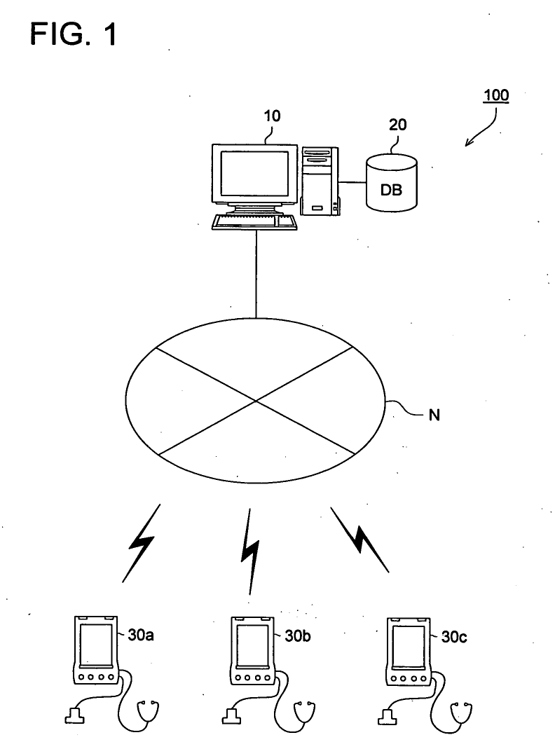

In FIG. 1, a system structure of a medical examination support system 100 in the present embodiment is shown. As shown in FIG. 1, in the medical examination support system 100, a server 10 to which a data base (hereinafter, called DB: Database) 20 is connected, and a plurality of data processing terminals 30a-30c, are connected so that they can mutually send and receive the data through the communication network N. Hereupon, in FIG. 1, a system structure in which a server, a DB, 3 data processing terminals are connected, is shown, however, the number of installation sets and the installation positions are not specially limited.

The server 10 conducts the input output control of data stored in the DB 20. The DB 20 is structured by a large capacity memory, and in the DB 20, the data of the body sound collected in data processing terminals 30a-30c, and its additional information are stored.

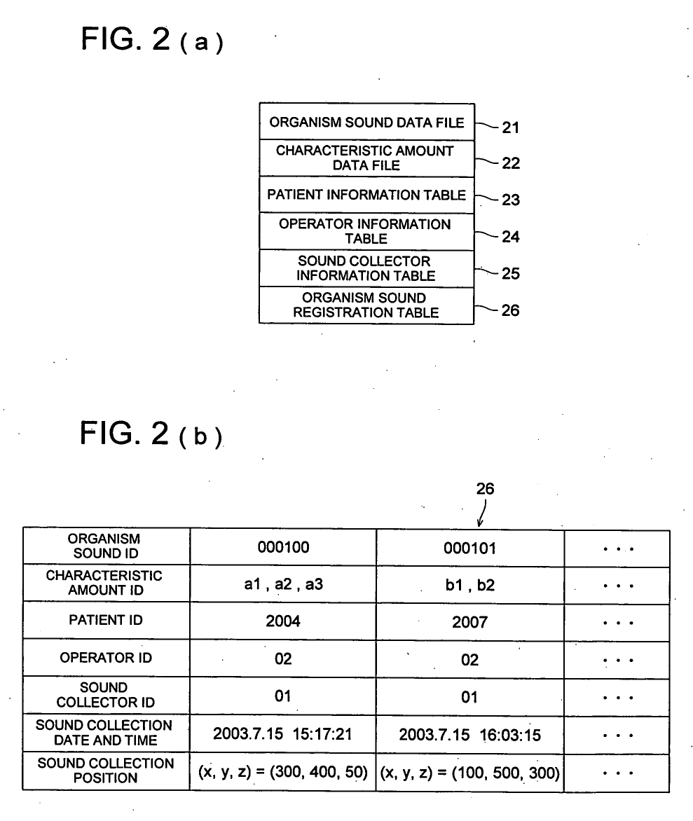

In FIG. 2(a), an example of the data structure of DB 20 is shown. As shown in FIG. 2(a), in the DB 20, an body-sound data file 21, characteristic amount data file 22, patient information table 23, operator information table 24, sound-collector information table 25, and body sound registration table 26 are accommodated.

In the body sound data file 21, the body sound data is accommodated. To each body sound data, the identification information for individually discriminating the body sound data (hereinafter, called body sound ID) is affixed, and in every body sound ID, the body sound data is accommodated.

In the characteristic amount data file, the characteristic amount data extracted from the organisms sound data is accommodated. The characteristic amount means each kind of characteristic amount relating to the body sound such as the frequency band of the body sound, amplitude, phase, when there is the abnormality in the body sound, the frequency band of the abnormal body sound, or the time at which the abnormal body sound is detected. In each characteristic amount data, the identification information for individually identifying the characteristic amount data (hereinafter, called the characteristic ID) is affixed, and for each the characteristic ID, the characteristic amount data is accommodated.

In the patent information table 23, the information relating to patents are accommodated. For each patient, the identification information for individually identifying the patient (hereinafter, called patient ID) is set, and for this each patient ID, each kind of information such as name of patient, age, sex, is accommodated.

In the operator information table, the information relating to the operator who sound-collects the body sound of the patient, is accommodated. For each operator, the identification information for individually identifying the operator (hereinafter, called operator ID) is set, and for each operator ID, each kind of information such as the name of operator, the attribute is accommodated. For example, the operator ID is set in such a manner that when the operator ID is shown by a code of “01”, the operator is A doctor, when the operator ID is shown by a code of “02”, the operator is B nursing person, and when the operator ID is shown by a code of “99”, the operator is patient itself.

Hereupon, when the code is a code showing that the operator is patient itself, (in the above example, the code of “99”), the operator information such as the name of operator, attribute is not accommodated, and necessary information may also be acquired from the patient information table 23. Further, a person table in which the patient information table 23 and the operator information table 24 are integrated, is provided, and in the person table, each kind of information such as the person ID as the identification information of the person, the name of the person, the attribute of the person such as doctor, nursing person, engineer, or patient, may also be accommodated. In this case, in the patient ID and operator ID of an body sound registration table, which will be described later, the person ID registered in the person table is accommodated.

In the sound collector information table 25, the information relating to the sound collector which collects the body sound, is accommodated. For each sound collector, the identification information for individually identifying the sound collector (hereinafter, called sound collector ID) is set, and for this sound collector ID, each kind of information such as the serial number of the sound collector, name of the maker is accommodated.

The body sound registration table 26 is a table in which the additional information of the collected body sound is registered, and as shown in FIG. 2(b), for each body sound ID, corresponding to the body sound ID, each information of the characteristic amount ID of the characteristic amount data extracted from the body sound data shown by the body sound ID, patient ID of the patient whose body sound is collected, operator ID of the operator who conducts the sound collection, sound collector ID of the sound collector used for the sound collection, sound collection date and time at which sound collection is conducted, and the sound collection position showing the position in which the sound collection is conducted (for example, it is shown by x, y, z coordinates), is accommodated.

Hereupon, in the characteristic amount ID, the characteristic amount ID of the characteristic amount data file 22 corresponding to the body sound ID is accommodated. Further, in the patient ID, any one of the patient ID registered in the patient information table 23, is accommodated, and in the operator ID, any one of the operator ID registered in the operator information table 24 is accommodated. Similarly, in the sound collector ID, any one of the sound collector ID registered in the sound collector information table 25 is accommodated.

Further, in the present embodiment, an example in which the sound collection position information is shown by the xyz-coordinates is described, however, when it is the information in which the sound collected position can be discriminated, it is not limited to this, by the information showing the region (for example, (the chest front), (chest back), (abdominal region), (waist)) and by the information showing the further detailed position (for example, (upper right part), (lower left part), (lower right part)), it may be expressed. Further, a sound collection position table in which the information showing the region for each sound collection date ID and the detailed position information in the region are registered correspondingly to the sound collection position ID for identifying the sound collection position, is separately provided, and in the body sound table 26, the sound collection position ID may also be registered instead of the sound collection information and it may also be other embodiments.

In the example shown in FIG. 2(b), for the body sound data of the body sound ID “000100”, the characteristic amount data “a1”, “a2”, “a3” are extracted from the body sound data, and the patient ID of the patient whose body sound is collected, is “2004”, the operator ID of the operator is “02”, the sound collector ID of the sound collector is “01”, the sound-collection date and time is “2003. 7. 15 15: 17: 21”, and the sound-collection position is a position of “(x, y, z)=(300, 400, 50)”, are registered.

Next, data processing terminals 30a-30c will be described. The data processing terminals 30a-30c are terminal apparatus by which the body sound data collected from the patient is data-processed, and the sound reproduction or its time-waveform is displayed. In the present embodiment, the data processing terminals 30a-30c are described as portable terminals, however, they may also be fixedly installed computer terminal apparatus.

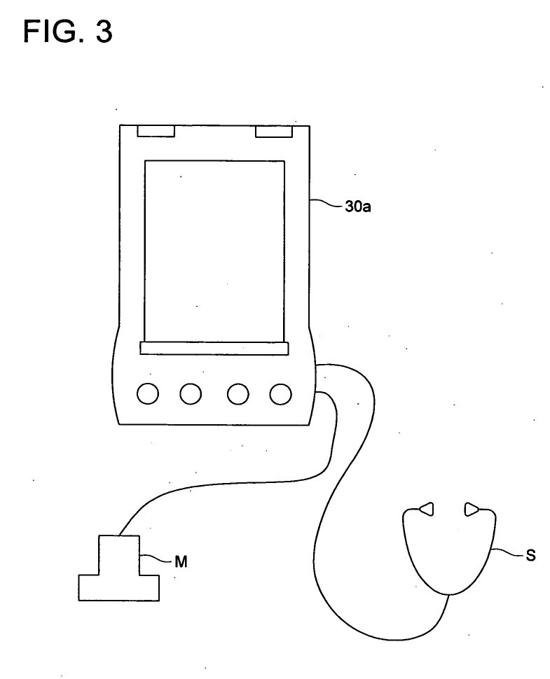

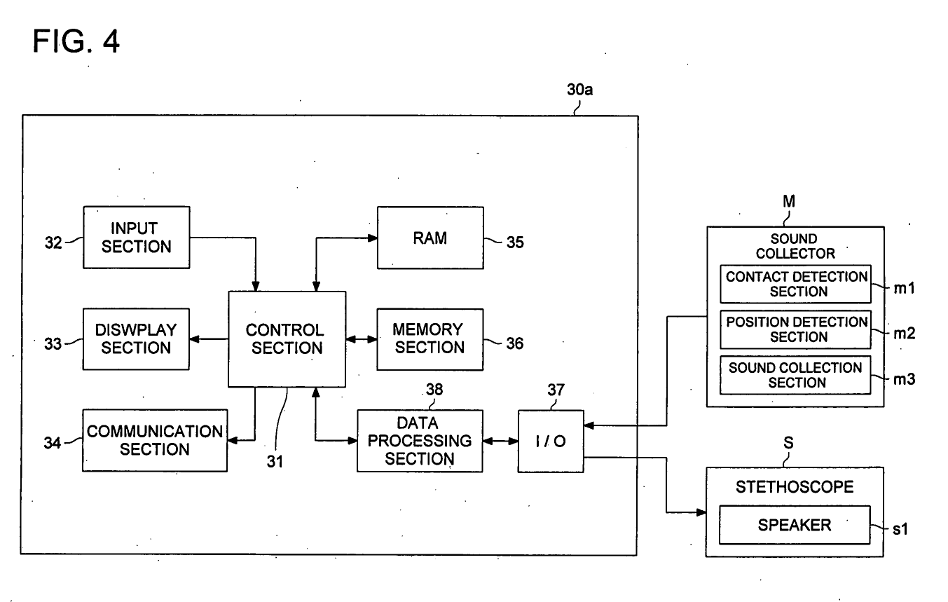

Referring to FIG. 3 and FIG. 4, the structure of the data processing terminals 30a-30c is described, and even when, in the data processing terminals 30a-30c, the appearance or application function is different, because the base structure is the same, the structure will be described on behalf of the data processing terminal 30a. FIG. 3 is a sketch drawing of the data processing terminal 30a. As shown in FIG. 3, the data processing terminal 30a is connected to the sound-collector M and stethoscope S.

In FIG. 4, the inner structure of the data processing terminal 30a is shown. As shown in FIG. 4, the data processing terminal 30a is structured by providing with: a section control section 31; input section 32; display section 33; communication section 34; RAM (Random Access Memory) 35; memory section 36; I/O (Input Output) section 37; and data processing section 38, and through the I/O section 37, the sound-collector M, and stethoscope S are connected.

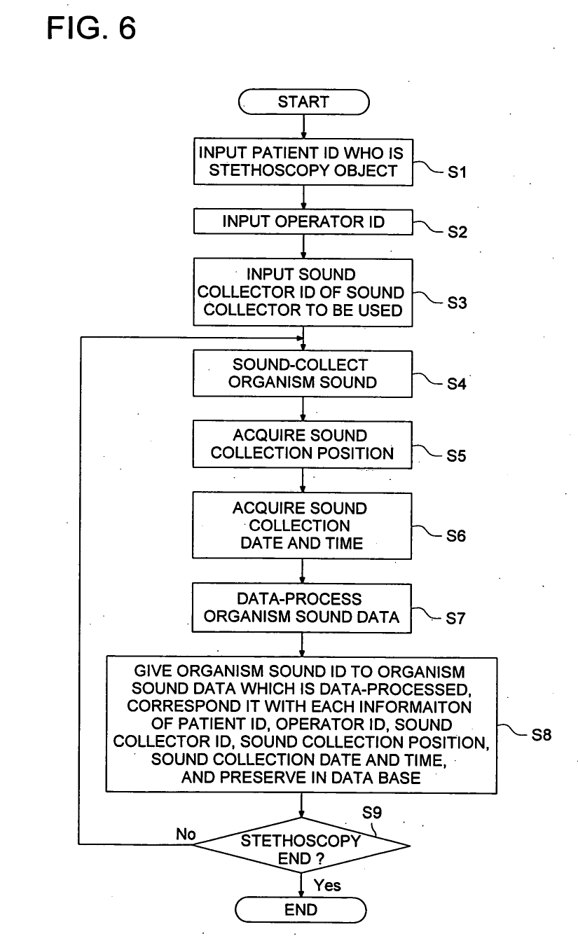

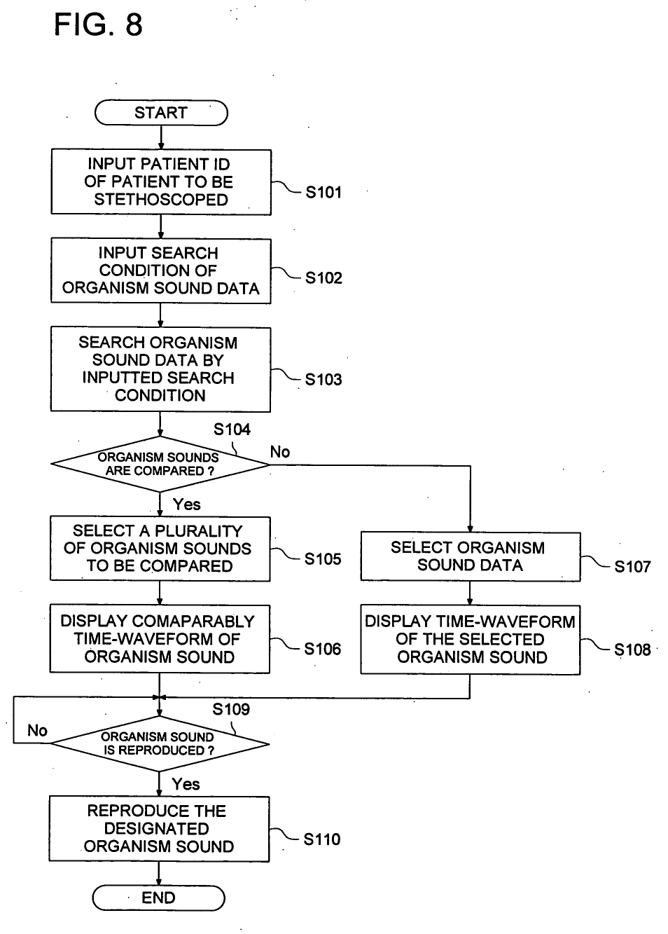

The control section 31 is structured by CPU (Central Processing Unit), and develops each kind of program stored in the memory section 36 into RAM 35, and by cooperation of the program, generalizingly controls the processing operations. Hereupon, in this program, the program according to the present invention is inhered, and the body sound preservation processing routine shown in FIG. 6, and the body sound reproduction processing routine shown in FIG. 8, are included.

The control section 31 gives, when the body sound data is generated by the data processing section 38 in the body sound preservation processing, the body sound ID to the body sound data. At this time, it acquires the information of the sound collection date of the sound collector M, and acquires the information of the sound collection date and time of the body sound.

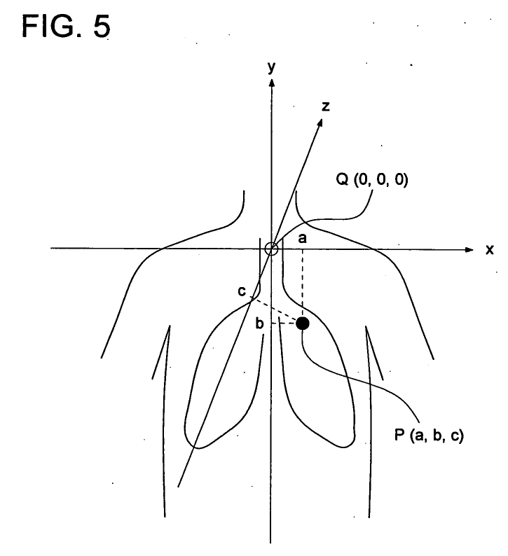

The information of the sound collection position is acquired when the sound collection position of the sound collector M according to the detection signal is calculated, based on a detection signal of the angular acceleration and acceleration inputted from a position detection section m2 of the sound collector M. Referring to FIG. 5, a detection method of the sound collection position will be described below. In the detection of the sound collection position, initially, the initial position is set. For example, as shown in FIG. 5, the sound collector M is brought into contact with the initial position (herein, the initial position is set between the collarbones of the patient. Q position shown in FIG. 5), previously set before the sound-collection is conducted, and the detection signal of the angular acceleration and the acceleration in the contacted position is acquired from the sound collector M. Then, the position at which this detection signal is acquired, is set to the initial position, that is, the origin of x y z coordinate. After the initial position is set, according to the detection signal of the angular acceleration and the acceleration inputted from the sound collector M, x y z coordinate showing the relative position in which how much distance it is moved from the initial position, is calculated, and this is made the sound collection position information. That is, by the position detection section m2 of the sound collector M, and control section 31, the position detection means can be realized.

Further, the control section 31 has a timing function using the clock signal, and the date and time at which the body sound data is generated, are timed by the timing function, and information of the sound collection date and time is acquired. That is, the timing means can be realized by the control section 31.

The control section 31 stores the body sound data generated by the data processing section 38 in DB 20 through the server 10. When the data is stored, together with the patient ID, operator ID, sound collector ID, which are inputted as the additional information of the body sound through the input section 32, each kind of information of acquired sound collection date and time, sound collection position is written in the body sound registration table 26 corresponding to the body sound ID. That is, when the control section 31 stores the body sound data and its additional information in the DB 20 according to the body sound preservation processing program, the data preservation means can be realized.

Further, in the body sound reproduction processing, on the basis of a search keyword such as the patient ID, operator ID inputted through the input section 32, the corresponding body sound data is searched from DB 20 and acquired. That is, when the control section 31 acquires the body sound data from DB 20 according to the body sound reproduction processing program, an acquire means can be realized.

The input section 32 is structured by providing with key group such as numeric keys or character keys, each kind of function keys, and a touch panel integrated with the display section 33, and the operation signal corresponding to the operated key is outputted to the control section 31. That is, when the patient ID, operator ID which are registered in DB 20 as the additional information of the body sound through the input section 32 are inputted, the input means can be realized.

The display section 33 is structured by providing with an LCD (Liquid Crystal Display), and displays each kind of display information such as the time waveform of the body sound data, each kind of operation image plane, and the processing result by the control section 31. That is, the body sound display means and the comparison result display means can be realized by the display section 33.

The communication section 34 is structured by providing with an interface or modem to connect a communication terminal such as the mobile phone or PHS (Personal Handy-Phone System), and a communication control section, and at the time of communication, the communication operation of the communication terminal is controlled by the communication control section, and the sending and/or receiving of the data is conducted to the external apparatus on the communication network N. Hereupon, without using the communication terminal, the communication may also be conducted by providing with a wireless LAN (Local Area Network) card.

The RAM 35 forms a work area which temporarily stores an each kind of program executed by the control section 31 and the data concerning to these programs.

The memory section 36 is structured by semiconductor memory, and stores, other than the system program, the body sound preservation processing program, the body sound reproduction processing program, and the data processed in each program.

The I/O section 37 is an interface to connect the data processing terminal 30a to the sound collector M and stethoscope S, and sends a sound signal of the body sound inputted from the sound collector M to the data processing section 38, and sends the analog sound signal inputted from the data processing section 38 to the stethoscope S. Hereupon, I/O 27 and the data processing section 38 are connected by a plurality of wires, and the simultaneous reciprocal sending of the signal can be conducted.

The data processing section 38 conducts sampling the analog sound signal inputted from the sound collector M through the I/O section 37 by a predetermined sampling frequency, and generates a digital body sound data. Further, corresponding to the direction from the control section 31, it converts the body sound data which is a reproduction object into an analog sound signal, and outputs to the stethoscope S through the I/O 37. Hereupon, the data processing section 38 amplifies the sound signal inputted through the I/O section 37 corresponding to the direction from the control section 31, and can also send to the stethoscope S.

Further, the data processing section 38 conducts each kind of data processing such as a filter processing for removing a noise from the body sound on the acquired body sound data, an FET (Fast Fourier Transform) processing for separating the normal body sound and the abnormal body sound from the time waveform data of the frequency spectrum of the body sound, and the characteristic amount extraction processing for extracting the characteristic amount data of the body sound from the time waveform of the body sound data.

When a pulmonary sound is taken as an example, the pulmonary sound is classified into a normal breath sound and a sub noise in which abnormality is found, and as the sub noise, a moist rale, crepitant rale, whistle sound (Ra-sound), snore sound, are listed.

In FET processing, by FET-processing the time waveform data of the pulmonary sound, the amplitude spectrum, phase spectrum, power spectrum are calculated, and when a local dispersion value calculated to the power spectrum does not exceed a previously set threshold value (for example, a threshold value for judging a continuous Ra-sound), the amplitude spectrum is discriminated as the spectrum of the normal pulmonary sound, and when the value exceeds the threshold value, the amplitude spectrum is discriminated as the spectrum of the continuous Ra-sound. When the inverse FET processing is conducted on the amplitude spectrum discriminated in this manner, the time waveform data corresponding to the normal pulmonary sound and the time waveform data corresponding to the continuous Ra-sound can be acquired.

Further, in the characteristic amount extraction processing, when the abnormality of Ra-sound is found in the time waveform data of the normal body sound discriminated in the above FET processing, or in the body sound, each kind of characteristic amount relating to the body sound such as the frequency band of the body sound, breathing time, time at which abnormal body sound is detected, is extracted from the time waveform data of the abnormal body sound.

Hereupon, the data processing section 38 may also conduct the data processing on-line by using DSP (Digital Signal Processor), or the body sound data is stored once in RAM 35 and after that, the stored body sound data may also be data processed on off-line. Further, for the data processing, the data processing may also be conducted by using the program or by using the hardware. Further, a part or the whole of such a data processing may also be processed, not by the data processing terminal 30a, but by a server 10.

The sound collector M is a sound collection means structured by providing a contact detection section m1, position detection section m2, sound collection section m3, and by the operator, when it is brought into contact with the medical examination part of the patient, the sound collection of the body sound is started, and the collected body sound signal is outputted to the data processing terminal 30a.

The contact detection section m1 is a section for detecting that the sound collector M is brought into contact with the body of the patient, and based on the detection signal outputted from this contact detection section m1, the start timing for starting the sound collection is determined. For example, an optical sensor is provided in the contact detection section m1, and by this optical sensor, when it is detected that the sound collector M is brought into contact with the patient, the sound collection is started by the sound collection section m3.

The position detection section m2 is a section for detecting the sound collection position by the sound collector M, and it is structured by including a gyro sensor for detecting the angular acceleration of the sound collector M, and an acceleration sensor for detecting the acceleration to the moving direction of the sound collector M. The detection signal of the angular acceleration and the acceleration detected by these sensors is outputted to the control section 31 of the data processing terminal 30a, and in the control section 31, a changing amount in the moving direction is calculated from the angular acceleration, and when the acceleration detected by the acceleration sensor is accumulated, the moving speed of the sound collector M is calculated. That is, the relative position change of the sound collector M is calculated.

To the sound collection section m3, a condenser-microphone is applied, and the body sound of the patient is sound-collected, and the body sound is generated based on the body sound signal.

The stethoscope S is a sound reproduction means structured by providing with an earphone composed of a speaker s1, and reproduces the sound signal inputted from the data processing section 38.

Next, the operation in the present embodiment will be described. FIG. 6 is a flowchart for explaining the body sound preservation processing which is conducted in the data processing terminals 30a-30c. This body sound preservation processing is a processing by which the body sound data collected from the medical examination part of the patient is made to be in the data base.

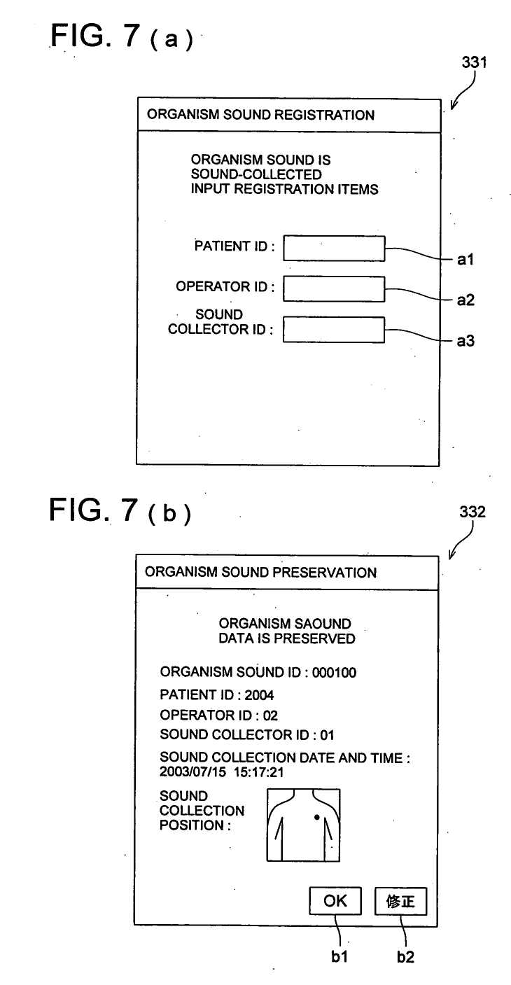

In the body sound preservation processing shown in FIG. 6, initially, the registration image plane 331 (refer to FIG. 7(a)) is displayed on the display section 33, and the registration information of the body sound to be sound-collected is inputted. As shown in FIG. 7(a), on the registration image plane 331, an input area a1 of the patient ID of the patient to be stethoscoped, an input area a2 of the operator ID of the operator, and an input area a3 of the sound collector ID of the sound collector M to be used for the sound collection are displayed.

In this registration image plane 331, when the patient ID is inputted through the input section 32 (step S1), the information of the patient ID operation-inputted is temporarily stored in RAM 35. Next, when the operator ID is inputted (step S2), the sound collector ID is inputted (step S3), the information of inputted operator ID and sound collector ID is made corresponded to the previously inputted patient ID, and temporarily stored in RAM 35.

Hereupon, it is preferable that the input of the patient ID is conducted in such a manner that a part or the whole of item information (for example, the patient name and the patient ID) of the patient information table is acquired from DB 20, and a part or the whole of the acquired patient information (for example, only the patient name) is displayed as choices on the display section 33, and the patient ID corresponding to the patient information selected from these choices is inputted.

In the operator ID and sound collector ID, a structure in which respective default values are stored and when particularly there is no change input, the default values are inputted, is preferable. Hereupon, this default value is preferable when, for example, a value inputted at the time of the initial setting of the data processing terminal or a value which is change-set after that, is registered.

The operator, when the input of each registration information is completed, operates the sound collector M and brings the sound collector M into contact with the medical examination part of the patient. When the sound collector M is brought into contact with the medical examination part of the patient, the sound collection of the body sound by the sound collector M is started (step S4), and an analog signal of the collected body sound is inputted into the data processing section 38 through the I/O section 37. In the data processing section 38, the digital body sound data is generated from the inputted body sound signal. Hereupon, it may also be allowable that the inputted body sound signal is outputted soon to the stethoscope S and reproduced.

Further, as the position of the sound collector M to be sound-collected, the detection signal of the angular acceleration and the acceleration detected by the position detection section m2 is outputted to the control section 31 through the I/O section 37. In the control section 31, based on the detection signal of the angular acceleration and the acceleration, the relative position coordinate from the initial position of the sound collector M is calculated, and temporarily stored in RAM 35 as the sound collection position information of the body sound in which the detected position coordinate is inputted (step S5). Next, by the timer function of the control section 31, the information of the sound collection date and time of the body sound is acquired by the timer function of the control section 31, and temporarily stored in RAM 35 (step S6).

When the sound collection is completed and the operator separates the sound collector M from the medical examination part of the patient, in the sound collector M, the sound collection operation is stopped, and in the data processing section 38, the data processing of the body sound whose sound collection is completed is conducted (step S3). In the data processing, the filter processing, FET processing, characteristic amount extraction processing are included, and to the extracted characteristic amount, the characteristic amount ID is affixed by the control section 31. Hereupon, the data processing may also be conducted at the time of preservation, or the data processing is not conducted the time of preservation, but the yet-processed body sound data is temporarily stored in DB 20, and the body sound data is acquired after from the DB 20, and the data processing may also be conducted when the body sound data is reproduced.

When the data processing of the body sound data is completed, the body sound ID is affixed to the body sound data, and the preservation image plane 332 as shown in FIG. 7(b) is displayed on the display section 33. On this preservation image plane 332, each information of the patient ID, operator ID, sound collector ID, sound collection position and sound collection date and time, temporarily stored in RAM 35, is displayed. After the operator confirms the registration content, when the registration content is allowable, the operator presses OK-key b1. When there is an amendment item in the registration content, the operator presses an amendment-key b2, and the image plane moves to the amendment image plane (not shown) of the registration content.

In the preservation image plane 332, the OK-key b1 is pressed, as the body sound data and the additional information of the body sound data, each information of the patient ID, operator ID, sound collector ID, temporarily stored in RAM 35, and the sound collection position, sound collection date and time, acquired in the control section 31 is sent to the server 10 through the communication section 34. In the server 10, each kind of information of the body sound data, patient ID, operator ID, sound collector ID, sound collection position, sound collection date and time, is stored in DB 20 (step S8). When the characteristic amount data is calculated from the body sound data, the information of the characteristic amount data and the characteristic amount ID is corresponded to the body sound data, and stored in DB 20.

Next, it is judged whether the indication that the stethoscopy is ended, is inputted through the input section 32 (step S9). When the indication of the stethoscopy completion is not inputted, and the medical examination part is changed by the operator, and a detection signal of angular acceleration and the acceleration is newly inputted from the sound collector M (step 9: N), the sequence returns to step S4, and the sound collection of the body sound in the next examination part is repeated, and when the indication of the stethoscopy completion is inputted (step 9: Y), the present processing is ended.

Next, referring to FIG. 8, the body sound reproduction processing executed by data processing terminals 30a-30c, will be described. This body sound reproduction processing is a processing by which, when the body sound is reproduced, the time waveforms of a different plurality of body sound data of the same patient are comparably displayed corresponding to the indication, and the specified body sound is reproduced.

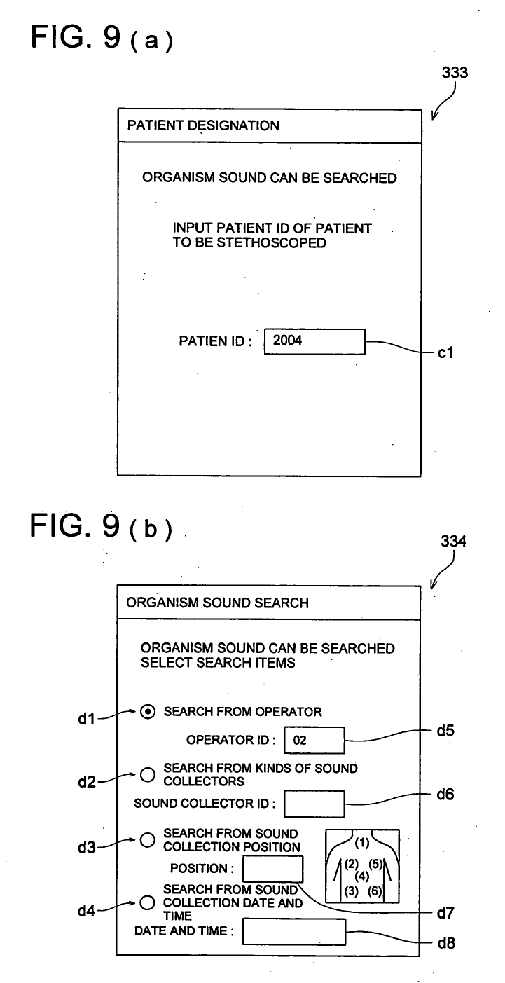

In the body sound reproduction processing shown in FIG. 8, initially, on the display section 33, a specification image plane 333 (refer to FIG. 9(a)) for specifying the patient who is a stethoscopy object is displayed, and the patient ID of the patient who is a stethoscopy object is inputted (step S101).

In the input area c1 of the patient ID provided on the specification image plane 333, when the patent ID is inputted through the input section 32, the body sound registration table 26 of DB 20 is referred by the control section 31, and each additional information of the body sound ID, characteristic amount ID, operator ID, sound collector ID, sound collection date and time, and sound collection position, is acquired. Then, based on this acquired, each additional information, a search image plane 334 (refer to FIG. 9(b)) for inputting the search condition of the body sound data, is displayed on the display section 33.

As shown in FIG. 9(b), on the search image plane 334, as search items, each kind of search items d1-d4 such as searches on the basis of the operator who conducts the sound collection operation of the body sound, searches on the basis of the kind of the sound collector used for the sound collection, searches on the basis of the sound collection position, that is, the medical examination part of the patient, or searches on the basis of the date and time at which the sound collection is conducted, are selectably displayed, and under the each search item, input areas d5-d8 for inputting the operator ID, sound collector ID, sound collection position, sound collection date and time, as the search keyword, are provided.

When any one of search items is selectively inputted through the input section 32, and the search keyword is inputted, the body sound data corresponding to the search keyword in the body sound registration table 216 is searched by the control section 31 (step S 103). For example, when the item of operator is selected as the search item, and the operator ID of “02” is inputted as the search keyword, from the body sound registration table 26 shown in FIG. 5(b), the body sound ID “000100” is searched as the body sound ID of the body sound data corresponding to the search keyword.

When the search is ended, a message asking whether the comparison of the body sound is conducted is displayed on the display section 33, and it is discriminated whether the comparison of the body sound is indicated corresponding to the input indication from the input section 32 (step S104).

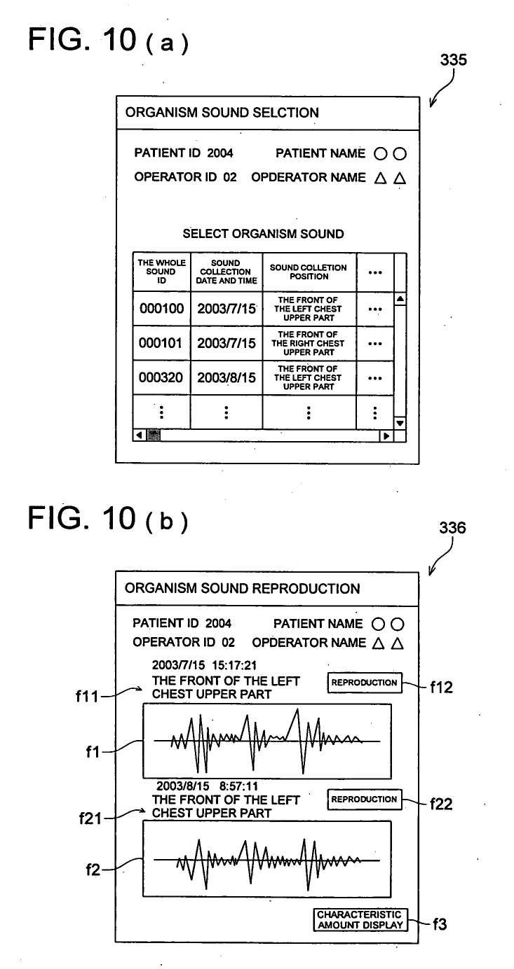

Initially, a case where the comparison of the body sound is indicated, will be described. When the comparison of the body sound is indicated (step S104: Y), the selection image plane 335 of the body sound as shown in FIG. 10(a), is displayed on the display section 33, and a plurality of body sounds to be compared, are selected (step S105). On the selection image plane 335, each additional information of the characteristic amount ID, operator ID, sound collector ID, sound collection date and time, and sound collection position, corresponding to the searched body sound ID is list-displayed for each body sound ID.

In this selection image plane 335, when a plurality of different body sounds are selected through the input section 32, the body sound data of the selected body sound is read from the body sound data file 21 of DB 20. Then, based on the read body sound data, the time waveforms of the frequency spectrum of the body sound are comparably displayed (step S106).

In FIG. 10(b), the reproduction image plane 336 on which the time waveforms of the body sounds are comparably displayed, is shown. As shown in FIG. 10(b), on the reproduction image plane 336, the time waveform f1 of the body sound sound-collected at the date and time of “2003/7/15, 15: 17: 21”, and the time waveform f2 of the body sound sound-collected at the date and time of “2003/8/15, 8: 57: 11”, are comparatively displayed in a column. Above each waveform f1, f2, the information f11, f21, of the sound collection date and time and the sound collection position are displayed, and the reproduction keys f12, f22, for inputting the reproduction indication of the body sound of the time waveform, are displayed.

Further, below the image plane, the characteristic amount display key f3 for indicating the translation to the data display of the characteristic amount extracted from the body sound by the data processing, is displayed. When this characteristic amount display key f3 is pressed, the characteristic amount data corresponding to the body sound which is comparatively displayed is read from DB 20, and the concerned characteristic amount data is displayed on the display section 33.

Next, on the reproduction image plane 336, the reproduction keys f12, f22 are pressed, and it is discriminated whether the reproduction of the body sound is indicated (step S109), and when the reproduction of the body sound is indicated (step S109: Y), the sound signal of the body sound indicated by the reproduction keys f12, f22, is outputted to the stethoscope S, and in the stethoscope S, the body sound is reproduced (step S110), and the present processing is completed.

Next, in step S104, the case where the comparison of the body sound is not indicated, will be described. When the comparison of the body sound is not indicated (step S104; N), the search image plane 334 of the body sound shown in FIG. 9(b), the selection image plane 335 of the body sound shown in FIG. 10(a), are successively displayed on the display section 33, the selection of the body sound which displays the time waveform, or which reproduces the time waveform, is conducted (step S107). Next, the body sound data of the selected body sound is read from DB 20, and the body sound data of the concerned selected body sound is read from the body sound data file 21 of DB 20, and its time waveform is displayed on the display section 33 (step S108).

Because, in this time waveform display, the time waveforms displayed in the reproduction image plane 336 of the body sound shown in FIG. 10(b), are not plural, but only becomes one, particularly, its image plane example is not shown. That is, the time waveform of the selected one body sound, sound collection date and time, sound collection position, and the reproduction key for indicating the reproduction of the body sound, are displayed.

Then, when the reproduction key is pressed, and the reproduction of the body sound is indicated (step S109: Y), the sound signal of the body sound indicated by the reproduction key is outputted to the stethoscope S, and in the stethoscope S, the body sound is reproduced (step S119), and the present processing is completed.

As described above, when the body sound data is stored in DB 20, because, to the body sound data, each kind of information of the patient ID, sound collection date and time information, and sound collection position information is corresponded and stored in DB20, even when the body sound is stethoscoped again after, or the operator and stethoscope person are different, the stethoscope person can easily grasp an attribute of the body sound such as the body sound of which patient the body sound to be reproduced is, and at which date and time the body sound of which part is collected.

Further, because the operator ID is stored in DB 20 as the additional information of the body sound, when the body sounds collected by the same operator are compared at the time of the stethoscopy, the stethoscopy considering the operation characteristic different depending on the operator such as a personal habit of the sound collection operation of the operator or the sound collction time, can be conducted. Further, because the sound collector ID is made the additional information of the body sound data, when the body sounds collected by the same sound collector are compared, the stethoscopy considering the sound collection characteristic different depending on the sound collector such as the instrument member or structure of the sound collector M, can be conducted.

Further, when the time waveform of the body sound is displayed, because a plurality of time waveforms of different body sounds can be comparably displayed, the time waveforms can be compared in various combinations such as the past and present, left chest and right chest, normal body sound and abnormal body sound separated from the body sounds, and the stethoscopy person can easily grasp the aging change such as the progressing condition or recovery condition, efficacy condition of medicine.

Further, when the body sound is reproduced or its time waveform is displayed, because the desired body sound data can be searched from DB 20, making the patient ID, operator ID, sound collector ID, sound collection position, sound collection date and time as the keywords, the stethoscope person can acquire the desired body sound data by an easy operation, and can improve the work efficiency at the time of stethoscopy.

The description content in the present embodiment, is a suitable example of the medical examination support system 100 to which the present invention is applied, and the invention is not limited to this.

For example, in the description described above, a structure in which the data processing terminals 30a-30c are portable terminals, and the data processing terminals 30a-30c and DB 20 are connected through the communication network N, is applied, however, a structure in which the data processing terminals 30a-30c are terminal apparatus which are fixedly installed, and DB 20 is housed, may also be applied. Further, the server 10 and DB 20 are made an integrated structure, however, a structure in which the server 10 and DB 20 are connected through the communication network N, may also be applied.

Further, in the body sound reproduction processing, the example in which the body sound data stored once in DB 20 is acquired and its time waveforms are comparably displayed is described, however, it is not limited to this, but the time waveform of the body sound data just generated before stored in DB 20 may also be comparably displayed or reproduced. In this case, a time in which the body sound data is read from DB 20 can be omitted, and the comparison of the body sounds can be conducted in real time.

Further, in the example described above, an example in which the time waveforms of body sounds are comparably displayed is described, however, it is not limited to this when the difference of each of body sounds can be visually compared, and for example, the characteristic amount of the body sound data which are comparative objects is numerically expressed and displayed, and based on the body sound data, a message in which the characteristic of each body sound, the name of assumed disease are made into a sentence, may also be displayed.

Further, the structure in which the body sounds whose time waveform are comparably displayed are reproduced in the order of designation, and each body sounds can be visibly compared, is applied, however, it is not limited to this, but for example, when 2 different body sounds are selected as the comparative object, the body sound of one hand of the comparative objects is outputted to the right ear-phone of the stethoscope S, the body sounds of the other hand is outputted to the left ear-phone, and they are simultaneously reproduced, and the body sounds of the comparative objects may also be possible to be simultaneously compared.

Further, when the body sounds are compared, the stethoscope person previously sets the comparison point, and when the time waveform which is a comparative object is displayed, the comparison point is marker-displayed to the time waveform, and in the case where the body sound which is a comparative object, is reproduced, when it reaches the comparative point during the reproduction of the body sound, an attention sound showing the comparative point may also be reproduced. Hereby, the point to be compared becomes clear, and the stethoscope person has an easy medical examination.

Further, the data processing section 38 compares a plurality of body sound data, and detects a point whose difference is large, in the frequency distribution or volume, change interval of the sound, and the comparison point may also be shown by making this as comparison result.

Other than that, also relating to the detailed structure and the detailed operation of the medical examination support system 100 in the present embodiment, it can be appropriately changed within the scope without departing the sprit of the present invention.

### INDUSTRIAL APPLICABILITY

According to the inventions written in items 1, 11, 21, because the body sound data of the patient is corresponded to the identification information of the patient and is stored in the data base, and by using the identification information of the patient, the body sound data corresponding to the concerned patient is acquired from the data base, when the body sound data stored in the data base is read and stethoscoped, the patient to be stethoscoped can be indicated, even when there are a plurality of patients to be stethoscoped, it can be judged to which patient the body sound belongs. Accordingly, a mistake of the body sound data of the patient can be prevented, and because the comparison result of a plurality of adequate body sounds is displayed, the highly reliable medical examination support can be conducted.

According to the inventions written in items 2, 12, 22, because a plurality of different body sound data are comparably displayed, the stethoscope person can visually compare the body sound data, for example, in the past and the present, the body sound data in the left chest and the right chest, and the progress-condition or the recovery condition of the disease can be judged.

According to the inventions written in items 3, 12, 23, because the data processing terminal compares a plurality of different body sound data and its comparison result is outputted, the calculation load on the data base side is decreased.

According to the inventions written in items 4, 14, 24, because a plurality of body sound data of the same patient are acquired, and the medical examination support can be conducted, it contributes the efficiency increase of the medical examination.

According to the inventions written in items 5, 15, 25, because the body sound data is acquired from the data base and the body sound is reproduced, the stethoscope person can stethoscope the past body sound stored in the data base. Accordingly, when the body sounds desired to compare, such as the body sound of the past and the present, are successively reproduced, the auditive comparison can be conducted.

According to the inventions written in items 6, 16, 26, because, as the additional information of the body sound data, the identification information of the sound collection means is stored in the data base, it can be easily judged that, by which sound collection means, the body sound data is sound-collected. For example, the correspondence that the body sound data relating to the sound collection means in which the nonconformity is discovered in the future, is not used for the stethoscopy, can be taken, and the adequate support of the medical examination can be conducted.

According to the inventions written in items 7, 17, 27, because the identification information of the operator as the additional information of the body sound data is stored in the data base, on the basis of the identification information of the operator, the body sound data can be acquired from the data base, and, for example, when the body sound data are compared, the body sound data sound-collected by the same operator is acquired, the medical examination considering the operation characteristic which is different depending on the operator, can be conducted.

According to the inventions written in items 8, 18, 28, because the information of the sound collection position of the body sound as the additional information of the body sound data, is stored in the data base, the stethoscope person can easily grasp the body sound, of which medical examination part it is.

According to the inventions written in items 9, 19, 29, because the information of the sound-collection date and time is stored in the data base as the additional information of the body sound data, the stethoscope person can easily grasp the body sound, when it is sound-collected.

According to the inventions written in items 10, 20, 30, because, on the basis of the additional information, the body sound data corresponding to the additional information is acquired, the stethoscope person indicates any one of the identification information of the sound collection means, the identification information of the operator, information of the sound collection position, information of the sound collection date and time, and can easily acquire the body sound datat desired to be strthoscoped. Accordingly, the desired body sound data can be easily searched, and the improvement of the medical examination efficiency can be intended.

1-30. (canceled)

31. A medical examination support system, comprising:
a sound collecting section to collect body sounds of a patient to generate body sound data;
a data processing terminal to conduct processing the body sound data of the patient; and
a data base connected to the data processing terminal;
wherein the data processing terminal comprises
an input section to input identification information of the patient who is a target of stethoscopy;
a data storing section to correlate the body sound data of the body sounds collected by the sound collecting section with the identification information of the patient and to store the body sound data in correspondence with the identification information in the data base;
an acquiring section to acquire the body sound data corresponding to the inputted identification information of the patient from the data base; and
a comparison result showing section to show a comparison result in which a plurality of different body sound data are compared.

32. The medical examination support system described in claim 31, wherein the data processing terminal comprises a body sound showing section for showing a plurality of different body sound data visually and comparably.

33. The medical examination support system described in claim 31, wherein the data processing terminal comprises a comparing section to compare a plurality of different body sound data and for outputting the comparison result to the comparison result showing section.

34. The medical examination support system described in claim 32, wherein the plurality of different body sound data are body sound data of a specific patient and include the body sound data acquired by using the identification information of the specific patient from the data base.

35. The medical examination support system described in claim 31, further comprising:
a sound reproducing section to reproduce the body sound based on the body sound data acquired by the acquiring section.

36. The medical examination support system described in claim 31, wherein, in the data processing terminal, the input section inputs the identification information of the sound collecting section when the body sounds are collected by the sound collecting section, and the data storing section stores the identification information of the sound collecting section inputted as the additional information of the body sound data in the data base.

37. The medical examination support system described in claim 31, wherein, in the data processing terminal, the input section inputs the identification information of an operator who conducts the sound collecting operation of the body sound when the body sounds are collected by the sound collecting section, and the data storing section stores the identification information of the operator inputted as the additional information of the body sound data in the data base.

38. The medical examination support system described in claim 31, further comprising:
a position detecting section to detect the sound collection position at which the sound collection is conducted by the sound collecting section, and in which the data storing section of the data processing terminal stores the information of the sound collection position detected as the additional information of the body sound data in the data base.

39. The medical examination support system described in claim 31, wherein the data processing terminal comprises a timer section to count date and time at which sound-collection is conducted by the sound collecting section and the data storing section stores the information of the sound collection date and time counted by the timer section as the additional information of the body sound data in the data base.

40. The medical examination support system described in claim 36, wherein, in the data processing terminal, the input section inputs with designation any one of the additional information of the identification information of the sound collecting section, identification information of the operator, information of the sound collection position, information of the sound collection date and time when the body sound data is acquired from the data base, and wherein the acquiring section acquires the body sound data corresponding to the additional information inputted with designation by the input section from the data base.

41. A data processing terminal, comprising:
an input section to input identification information of the patient who is a target of stethoscopy;
a data storing section to correlate body sound data of body sounds collected by a sound collecting section with the identification information of the patient and to store the body sound data in correspondence with the identification information in the data base;
an acquiring section to acquire the body sound data corresponding to the inputted identification information of the patient from the data base; and
a comparison result showing section to show a comparison result in which a plurality of different body sound data are compared.

42. The data processing terminal described in claim 41, further comprising:
a body sound showing section for showing visually and comparably a plurality of different body sound data.

43. The data processing terminal described in claim 41, further comprising:
a comparing section for comparing a plurality of different body sound data, and for outputting the comparison result to the comparison result showing section.

44. The data processing terminal described in claim 42, wherein the plurality of different body sound data are body sound data of a specific patient and include the body sound data acquired from the data base by using the identification information of the concerned patent.

45. The data processing terminal described in claim 41, wherein the body sounds are reproduced by the sound reproducing section, based on the body sound data acquired by the acquiring section.

46. The data processing terminal described in claim 41, wherein the input section inputs the identification information of the sound collecting section when the body sounds are collected by the sound collecting section and the data storing section stores the identification information of the sound collecting section inputted as the additional information of the body sound data in the data base.

47. The data processing terminal described in claim 41, wherein the input section inputs the identification information of an operator who conducts the sound collecting operation of the body sounds when the body sounds are collected by the sound collecting section and the data storing section stores the identification information of the operator inputted as the additional information of the body sound data in the data base.

48. The data processing terminal described in claim 41, wherein the data storing section stores the information of sound-collection position detected as the additional information of the body sound data in the data base when the sound-collection position is detected by a position detecting section to detect the sound collection position at which the sound-collection is conducted by the sound collecting section.

49. The data processing terminal described in claim 41, further comprising:
a timer section to count date and time at which the sound-collection is conducted by the sound collecting section, wherein the data storing section stores the information of sound-collection date and time counted by the timer section as the additional information of the body sound data in the data base.

50. The data processing terminal described in claim 46, wherein the input section inputs with designation any one additional information of the identification information of the sound collecting section, identification information of the operator, information of the sound collection position, information of the sound collection date and time when the body sound data is acquired from the data base, and wherein the acquiring section acquires the body sound data corresponding to the additional information inputted with designation by the input section from the data base.

51. A data processing program for making a computer to realize functions, comprising:
a data preserving function to correlate body sound data of body sound collected by a sound collecting section with identification information, inputted by a input section, of a patient who is a target of stethoscopy and to store the body sound data in correspondence with the identification information in a data base;
an acquiring function to acquire the body sound data corresponding to the inputted identification information of the patient from the data base; and
a comparison result showing function to show a comparison result in which a plurality of different body sound data are compared.

52. The data processing program written claim 51, further comprising:
a function to show a plurality of different body sound data visually and comparably on a body sound showing section.

53. The data processing program written claim 51, further comprising:
a comparing function to compare a plurality of different body sound data and to output the comparison result to the comparison result showing function.

54. The data processing program written claim 52, wherein the plurality of different body sound data are body sound data of a specific patient and include the body sound data acquired from the data base by using the identification information of the specific patient.

55. The data processing program written claim 51, further comprising:
a sound reproducing function to reproduce the body sound by the sound reproducing section, based on the body sound data acquired by the acquiring function.

56. The data processing program written claim 51, wherein the data preserving function stores the identification information of the sound collecting section inputted as the additional information of the body sound data in the data base, in the case where the body sounds are collected by the sound collecting section when the identification information of the sound collecting section is inputted through the input section.

57. The data processing program written claim 51, wherein the data preserving function stores the identification information of an operator inputted as the additional information of the body sound data in the data base, in the case where the body sounds are collected by the sound collecting section, when the identification information of the operator who conducts the sound collecting operation of the body sounds, is inputted through the input section.

58. The data processing program written claim 51, wherein the data preserving function stores the information of the sound collection position detected as the additional information of the body sound data in the data base, when the sound collection position is detected by the position detecting section for detecting the sound-collection position at which the sound-collection is conducted.

59. The data processing program written claim 51, further comprising:
a timer function to count date and time at which the sound-collection is conducted by the sound collecting section, and in which the data preserving function stores the information of the sound collection date and time timed as the additional information of the body sound data in the data base.

60. The data processing program written claim 56, wherein the acquiring function, in the case where the body sound data is acquired the data base, when any one additional information of the identification information of the sound collecting section, identification information of the operator, information of the sound collection position, information of the sound collection date and time, is inputted with designation through the input section, acquires the body sound data corresponding to the additional information inputted with designation from the data base.
